(19) United States
(12) Patent Application Publication
(10) Pub. No.: US 2003/0136176 A1
(43) Pub. Date: Jul. 24, 2003

(54) Gas pressure/flow control and recovery system

(76) Inventor: Frank Ruiz, Greenwell Springs, LA (US)

Correspondence Address: Joseph J. Pophal, PARKER-HANNIFIN CORPORATION, 6035 Parkland Boulevard, Cleveland, OH, 44124-4141, US

(21) Appl. No.: 10/350,501

(22) Filed: Jan. 23, 2003

Related U.S. Application Data
(60) Provisional application No. 60/351,029, filed on Jan. 23, 2002.

(51) Int. Cl. G01N 33/00; G01N 37/00

(52) U.S. Cl. 73/23.2; 73/1.07

(57) ABSTRACT

A closed, gaseous fluid analyzing system includes a gas analyzer measuring cell that operates under substantially stable conditions by controlling both the pressure and flow rate of a plurality of differing gas streams while passing through the analyzer measuring cell. The plurality of gas streams are individually extracted and segregated while passing through the system, with the measuring cell being positioned between a flow controller and pressure regulator that cooperate to control upstream and downstream pressure and flow rate fluctuations in the gas streams. Methods of maintaining both substantially constant pressure and flow rate for the gas streams flowing through the analyzer measuring cell are also set forth.

### CROSS-REFEENCE TO RELATED CASES

The present application claims the benefit of the filing of U.S. Provisional Application Serial No. 60/351,029; filed Jan. 23, 2002.

### FIELD OF THE INVENTION

The present invention relates to a system and method of substantially isolating a gas analyzer measuring cell from upstream and downstream pressure and flow rate fluctuations that affect the gas streams passing therethrough.

### BACKGROUND OF THE INVENTION

Gases, which include effluent, exhaust, process types, and so forth, from both industrial and non-industrial applications are generally monitored to ensure that the concentration of certain constituents do not vary from predetermined limits. Gas analyzers are used to determine the concentrations of particular components, such as Oxygen, Carbon Dioxide, Carbon Monoxide, and so forth, in a gas sample. In the analysis of gases, it is well known that measurements must be performed under stable operating conditions. Variations in flow rates, temperatures and pressures can negatively affect the concentrations of the gas constituents that reach the analyzer. Even minor fluctuations will impair the functionality and thus effect the accuracy of the analyzer.

There are many types of analyzers being used today in both industrial and non-industrial applications. Almost all of these analyzers can be divided into two categories, “Continuous” and “Non-continuous”. Continuous analyzers require a continuous flow of the gas sample through the analyzer measuring cell. This produces a continuous measurement or analysis of the sample stream. Continuous analyzers are typically used in applications such as stack monitors, ambient air monitors, process control, and environmental monitors.

Non-continuous analyzers, or batch analyzers, generally operate on a timed cycle. Usually, the sample is introduced into the analyzer at the beginning of the cycle and the analyzing takes place during the remainder of the cycle. The cycle times can vary from one minute for a fast cycle, to an hour or more.

To maximize the analyzer's accuracy and reliability, both continuous and non-continuous types must be frequently calibrated. Generally, two gases are required to calibrate the analyzer. One is called the “zero” gas and the other is called the “calibration”, or “span” gas. First the “zero” gas is introduced into the analyzer and sufficient time is allowed for the analyzer to stabilize. The analyzer is then adjusted to output a zero reading. Thereafter, the known “span” gas is introduced and sufficient time is allowed for the analyzer to again stabilize. Finally, the analyzer is adjusted to reflect the concentration values of the known “span” gas. After this calibration, the analyzer is ready to receive the sample gas stream. As previously mentioned, for optimum accuracy, it is important that the analyzer measuring cell operate at a constant pressure, flow rate and temperature during both the calibration cycle and the analyzing operation.

The several noted gas sources used for the calibration procedure may have pressures and flow rates which can differ from those of the sample gas source. In fact, the pressures and flow rates of the several calibration gases can even fluctuate during the calibration procedure. These upstream pressure and flow rate differentials present a significant impediment for the measurement accuracy of the analyzer measuring cell.

The more serious problem with respect to the measurement accuracy of existing analyzers is that downstream gas backpressures must be relieved before reaching the analyzer measuring cell. Unlike prior art analyzer operating parameters, exhaust emissions from analyzers operated today can no longer be vented to atmosphere and must now be recaptured within the system and properly disposed of in an environmentally acceptable manner. Previously, when such emissions were vented directly to atmosphere, the system had a steady atmospheric downstream pressure. In today's environmentally conscious method of operation, this is no longer feasible, and backpressure fluctuations frequently translate back to the analyzer measuring cell, and will adversely affect its accuracy.

Prior art, such as U.S. Pat. No. 4,097,187 to Navarre, Jr., addresses the problem of differing upstream pressures and flow rates of the calibration and sample gases. Due to the less demanding environmental restrictions at the time of its invention, this reference does not consider the downstream pressures that can adversely affect the operation and accuracy of the analyzer measuring cell and vents its exhaust emissions directly to atmosphere. This is no longer acceptable.

Other prior art references, such as U.S. Pat. Nos. 5,756,360 and 6,200,819, both to Harvey et al., and related U.S. Pat. No. 5,968,452 to Silvis, are not directly related to the scope of the present invention but rather relate to the proper mixture, flow rate, and pressure of only the diluent gases utilized for the calibration procedure. Furthermore, these references are not concerned with the downstream pressure and flow rate fluctuations. Other examples of proper mixture, flow rate, and pressure regulation are set forth in U.S. Pat. No. 5,804,695 to Dageforde, and U.S. Pat. No. 5,239,856 to Mettes et al.

### SUMMARY OF THE PRESENT INVENTION

The present invention enables a gas analyzer measuring cell, within a closed gas analyzer system, to function under substantially stable conditions. This invention overcomes the obstacle of adverse upstream pressures and downstream backpressures that can occur in closed systems, and their effects on the accuracy of the analyzer's measurement by controlling that all gas streams have substantially the same pressure and flow rate while being analyzed as they pass through the analyzer measuring cell.

A feature of the present invention is to provide a pressure/flow control and gas recovery system in an apparatus for successively removing a sample of a flowing gas stream, analyzing the sample gas stream for at least one constituent thereof and thereafter returning said analyzed sample gas stream to a gas recovery system for disposal, the pressure/flow control and vent recovery system includes: a plurality of differing pressurized gas sources, having a zeroing and calibration gas stream in addition to the sample gas stream; an individual control valve operatively interconnected with each zeroing, calibration and sample gas stream for controlling their flow; a gas analyzer measuring cell, of either the continuously or the non-continuously operating type, successively operatively interconnected with the individual control valves and the gas recovery system; a pressure regulator, operatively interconnected with the gas analyzer measuring cell, for successively controlling the pressures of the zeroing, calibration and sample gas streams such that the controlled pressures are substantially constant upon the exits of the gas streams from the pressure regulator; and a flow controller, operatively interconnected with the gas analyzer measuring cell for successively regulating the zeroing, calibration and sample gas stream flow rates while flowing through the gas analyzer measuring cell, to a substantially constant value, and for substantially preventing backpressure variations from entering the gas analyzer measuring cell from the gas recovery system.

The previously noted system may further include a filter assembly, operatively interconnected with the sample gas stream, having a stream separator for separating the sample gas stream into a first sample gas stream, and filtering same, as well as a second sample gas stream which by-passes the filtering step and is operatively interconnected with the gas recovery system.

The previously noted system may additionally include, in lieu of the noted individual control valves, a stream switching manifold device operatively interconnected with the zeroing, calibration and sample gas streams for selectively segregating the gas streams, the stream switching manifold device having but one outlet.

The noted system may further include an adjustable needle valve, operatively interposed in the second sample gas stream, for varying the volume of the second sample gas stream prior to entering the gas recovery system. The flow controller is comprised of an additional needle valve operatively interconnected with a backpressure regulator, with the additional needle valve being adjustable for varying the volumes of the zeroing, calibration, and first sample gas streams.

In one embodiment of this invention, the pressure regulator is operatively interposed between the outlet of the individual control valves and the gas analyzer measuring cell, while in another embodiment of this invention, the pressure regulator is operatively interposed between the gas analyzer measuring cell and the gas recovery system. Similarly, the flow controller may be operatively interposed either between the gas analyzer measuring cell and the gas recovery system, or between the outlet of the individual control valves and the gas analyzer measuring cell.

Another feature of the present invention includes a method of maintaining both a constant pressure rate and a constant flow rate for the sample gas stream flowing through the gas analyzer measuring cell operatively interposed in a system between a flowing pressurized gas stream and the gas recovery system, the method including the steps of directing the sample gas stream, from the gas stream, into and through the analyzer measuring cell; controlling the value of the pressure of the sample gas stream for operating the gas analyzer measuring cell at a substantially constant value; controlling the value of the flow rate of the sample gas stream so that the flow rate remains at a substantially constant value while the sample gas stream passes through the gas analyzer measuring cell; analyzing the sample gas stream for at least one constituent thereof, while under substantially constant pressure and flow rate values, as the sample gas stream passes through the gas analyzer measuring cell; and directing the analyzed sample gas stream to the gas recovery system.

The previously noted method may include the initial steps of directing a zero gas stream into and through the gas analyzer measuring cell for stabilizing the operation of the cell; directing a calibration gas stream into and through the gas analyzer measuring cell for calibrating the cell; controlling the value of the pressures of the zero and calibration gas streams for operating the gas analyzer measuring cell at a substantially constant value; and controlling the value of the flow rates of the zero and calibration gas streams so that the flow rate remains at a substantially constant value while the zero and calibration gas streams pass through the gas analyzer measuring cell.

The method of this invention may further include the steps of segregating the zero, calibration and sample gas streams from each other before flowing to the gas analyzer measuring cell. In addition, the following steps may be included: directing the sample gas stream from the gas stream to a filter assembly; separating the sample gas stream into a first sample gas stream, and filtering same, and a second sample gas stream, thus by-passing the filtering step; directing the filtered first sample gas stream into and through the gas analyzer measuring cell; directing the by-passed second sample gas stream into the gas recovery system; and optionally varying the volume of the by-passed second sample gas stream prior to the entering of the by-passed sample gas stream into the gas recovery system.

As previously described, the features of the present invention serve to provide a unique, accurate gas analyzer system and apparatus as well as a method for operating an analyzer measuring cell for a closed analyzer system. Further features and advantages of the present invention will become apparent to those skilled in the art upon review of the following specification in conjunction with the accompanying drawings.

### BRIEF DESCRIPTION OF THE DRAWINGS

FIG. 1 is a schematic diagram of a pressure/flow control and gas recovery system for a gas analyzer measuring cell constructed in accordance with the present invention.

FIG. 2 is an enlarged view of the circled portion of FIG. 1 showing one arrangement of the pressure/flow controllers with the analyzer measuring cell.

FIG. 3 is a view, similar to that of FIG. 2, but showing a differing second arrangement of the pressure/flow controllers with the analyzer measuring cell.

### DETAILED DESCRIPTION OF THE PREFERRED EMBODIMENT

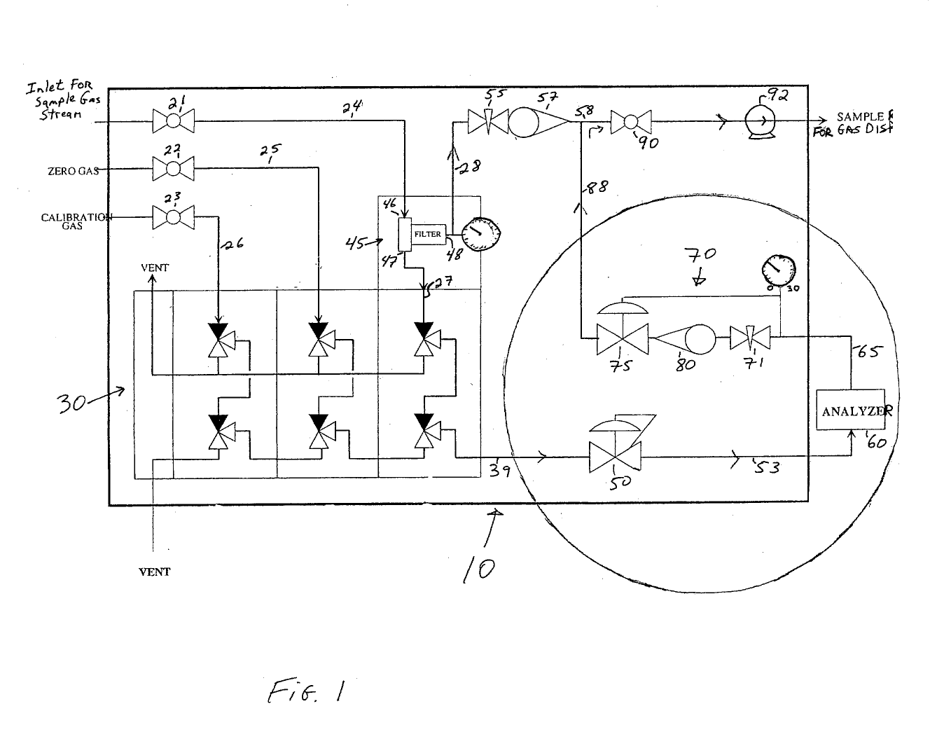

According to the present invention, FIG. 1 shows a first embodiment 10 of a flow diagram illustrating how a pressure/flow control and gas recovery system for a gas analyzer operates in order to analyze gases. System 10 is basically comprised of a series of inlet ball valves, 21-23, a stream switching system 30, an optional by-pass filter 45, a pressure-reducing regulator 50, an analyzer measuring cell 60, a flow controller 70, and a return valve 90. The present invention details how both the pressure and flow rate of gas streams through the analyzer measuring cell 60 are stabilized even with greatly varying inlet and outlet pressures.

Each inlet ball valve 21-23 is adaptable to a gas supply by means of an appropriate connector. A typical system for use with a gas analyzer utilizes at least three inlet gas lines: a calibration gas line, a zero gas line, and a sample gas line. The sample gas line, which emanates from a gas stream (not shown per se), is connected to a positive shut off control inlet ball valve 21, which controls the flow of the sample gas. It should be understood by one skilled in the art that the term gas stream, as used here and hereinafter, includes any type of gas stream, such as for example but not limited to: an effluent gas stream, an exhaust gas stream, or a process gas stream. Inlet ball valve 21 in turn is connected to by-pass filter 45 with a connecting line 24. By-pass filter 45 is an optional component of the stream switching system 30 that can be physically affixed thereto and which serves to remove possible undesirable particles in some sample gas streams that could be detrimental to the functioning of analyzer measuring cell 60. A predetermined percentage of the gas entering filter 45 passes through an internal filtering element (not shown) as a first sample stream while the remainder bypasses the filtering element as a second sample stream.

Filtered sample gas leaving filter 45 flows through line 27 into the stream switching system 30. The zero gas line is connected to inlet ball valve 22, which controls the flow of the zero gas with a positive shut off control, with inlet ball valve 22 being connected to system 30 by a connecting line 25. The calibration gas line is connected to inlet ball valve 23, which controls the flow of the calibration gas with a positive shut off control, with inlet ball valve 23 being connected to system 30 by means of a line 26. As will be described in more detail hereinafter, stream switching system 30 has multiple inlet flow streams, via connecting. lines 25, 26 and 27, but only a single outlet flow stream, as shown via a line 39.

Sample gas by-passed around the filtering element of filter 45 flows through a line 28 into a flow metering valve 55 that preferably includes a flow meter 57. This by-passed sample stream exits flow meter 57 through a line 58 and enters a return valve 90. A line 88 (to be described later) conveys the analyzed sample stream to line 58 and the reunited sample streams enter return valve 90 as a single stream.

The filtered or first sample gas stream that exits system 30 through outlet line 39 passes through pressure reducing regulator 50. This gas stream exits pressure reducing regulator 50 through a line 53 and enters analyzer measuring cell 60. Varying pressure streams enter regulator 50 and are reduced to a constant predetermined pressure suitable for analyzing the process stream. An example of a commercially available pressure-reducing regulator for use in this system is the Veriflo Model IR-5000, manufactured by The Parker Hannifin Corporation of Cleveland, Ohio. The Veriflo Model IR-5000, fully described in U.S. Pat. No. 4,807,849 to Morgan, which is fully incorporated herein by reference, is also assigned to the assignee of the present invention. This regulator can tolerate pressures as high as 3500 psig and is suitable for this application due to its ability to provide the required stability of the reduced exiting pressure (e.g. −3 to +30 psig) of the process stream. Other regulators can be used depending on the process stream pressure and the operating pressure of the analyzer measuring cell 60.

Analyzer measuring cell 60 determines the concentration of at least one particular component in the gas stream. A commonly used analyzer is the infrared absorption (IR) type. One example of such a commercially utilized continuous IR analyzer is the Vista Multiwave Photometer, available from the multi-national ABB Inc.. This style of analyzer is used for continuous chemical analysis of process streams and operates by passing an infrared energy light beam through a sample of process fluid. The IR energy is absorbed as it is passed through the process fluid and the pattern of wavelengths, or frequencies, absorbed identifies the molecules in the sample. In order for the analyzer to measure accurately and consistently, the pressure must remain constant. Another widely used analyzer is the paramagnetic oxygen analyzer which operates on the principle that the oxygen molecule has a strong affinity for a magnetic field. An example of such a commercially utilized continuous paramagnetic oxygen analyzer is the Xentra 4900 series Continuous Emissions Analyzer, available from Servomex International Ltd. in the U.K.. While the principle of operation is completely different, when referenced to the IR absorption type, the paramagnetic oxygen analyzer has the same requirement for a stable pressure.

The analyzed gas stream exits analyzer measuring cell 60 through a line 65 and enters flow controller 70. Flow controller 70 is comprised of two main interconnected components, a needle valve 71 and a backpressure regulator 75. Line 65 connects analyzer measuring cell 60 to needle valve 71. The gas stream flows through needle valve 71 and enters backpressure regulator 75. A flow meter 80 attached to needle valve 71 indicates the gas stream flow rate. Thereafter, the gas stream exits flow controller 70, passes through a line 88 and enters line 58 prior to entering return valve 90. Upon exit from return valve 90, the returned sample gas streams are routed to a gas recovery system (not shown per se) for disposal. The sample gas line 58 cannot be vented to atmosphere since the sample gas stream must be disposed of in an environmentally safe fashion, by being routed to the gas recovery system.

The operation of pressure/flow control and gas recovery system 10 will now be described. Each of the noted gases is sequentially introduced to the system as a gaseous stream through its respective inlet line while under pressure. The several gaseous streams typically operate at different pressures. In order to produce an accurate reading, it is important that analyzer measuring cell 60 operates at a constant pressure. It is also important that each gaseous stream has a constant flow rate while passing through analyzer measuring cell 60. The flow rate is of course directly proportional to the pressure of the gaseous stream.

Before the sample gas stream can be analyzed, analyzer measuring cell 60 must be accurately calibrated. This is accomplished in a known sequential manner with the zero gas and calibration gas streams. The zero gas stream is first introduced into the system through inlet ball valve 22. Typically, the zeroing gas is either air or nitrogen. Stream switching system 30 is previously configured so as to permit the passage of only the zeroing gas. Once analyzer measuring cell 60 has been stabilized with the zero gas, it is adjusted to output a zero reading.

The calibration or span gas stream is then introduced through inlet ball valve 23. Again switching system 30 is configured so as to permit the passage of only the calibration gas stream. Once analyzer measuring cell 60 has been stabilized with the calibration gas, it is adjusted to output a reading equal to the known concentration of the calibration gas. For example, if the known calibration gas is 9.82% oxygen, analyzer measuring cell 60 is adjusted to reflect a reading of 9.82% oxygen.

The sample gas stream is introduced to the system through inlet ball valve 21 after the calibration step is complete. The system can include the noted optional by-pass filter 45 which can take the form of a by-pass filter commonly used in the industry, e.g. a Balston cartridge filter type 95S, available from the Parker Hannifin Corporation of Cleveland, Ohio. The sample gas stream enters a filter bowl (not shown) of filter 45 through a filter inlet port 46. Only a small portion, e.g. less than 25%, of the incoming sample gas stream passes through the filter element. This filtered portion exits filter 45 through a filter outlet port 47 and flows into the stream switching system 30. The switching system 30 is designed so that filter 45 can be functionally affixed thereto and can, for example, take the form of the commercially available R-Max™ Stream Switching System, manufactured by the Parker Hannifin Corporation of Cleveland, Ohio. This R-Max™ Stream Switching System, fully described in copending U.S. pat. application Ser. No. 09/931,337, which is fully incorporated herein by reference and also assigned to the assignee of the present, invention, is a multi-functional system capable of switching various gas streams while preventing cross contamination of the streams. Any other stream switching system that segregates the streams and prevents cross contamination can be used.

As previously noted, the remaining unfiltered by-passed or second sample gas stream exits the filter bowl through by-pass exit port 48. This by-passing function serves three main benefits. First, a high flow rate passing through the filter reduces the transport time of the sample fluid. The transport time is defined as the time required for a sample fluid to travel from the process take-off point, e.g. the sample gas stream, (not shown), through a transport line into the inlet port of ball valve 21. Secondly, this unfiltered gaseous fluid generates a high flow rate which provides a continuous flushing, or purging, action of the filter bowl. Lastly, the life of the filter element is greatly extended because only a fraction of the total sample gas stream flow is filtered. This by-passed gas stream flows through line 28 into a flow metering valve 55 which provides a manual flow adjusting capability. Flow meter 57, attached to valve 55 provides a visual indication of a by-pass flow rate. This flow rate affects the transport time of the sample gas stream and too low of a flow rate will result in an unacceptable response time. Upon exiting metering valve 55, the by-passed sample gas stream joins the returned analyzed sample stream and both enter return valve 90 as a single stream.

As previously described, it is important that analyzer measuring cell 60 operates at a substantially constant pressure. Both upstream and downstream pressures, as well as flow rates, can affect the pressure at analyzer measuring cell 60. Upstream pressure fluctuations are common since different gases are introduced from various sources. For example, a calibration gas can be supplied from an individual tank. Depending on the amount of stored gas remaining in the tank, the pressure can change throughout its use. A more noticeable fluctuation in pressure occurs when one gas stream is switched to another gas stream. These varying pressures have to be regulated to a substantially constant pressure before reaching analyzer measuring cell 60.

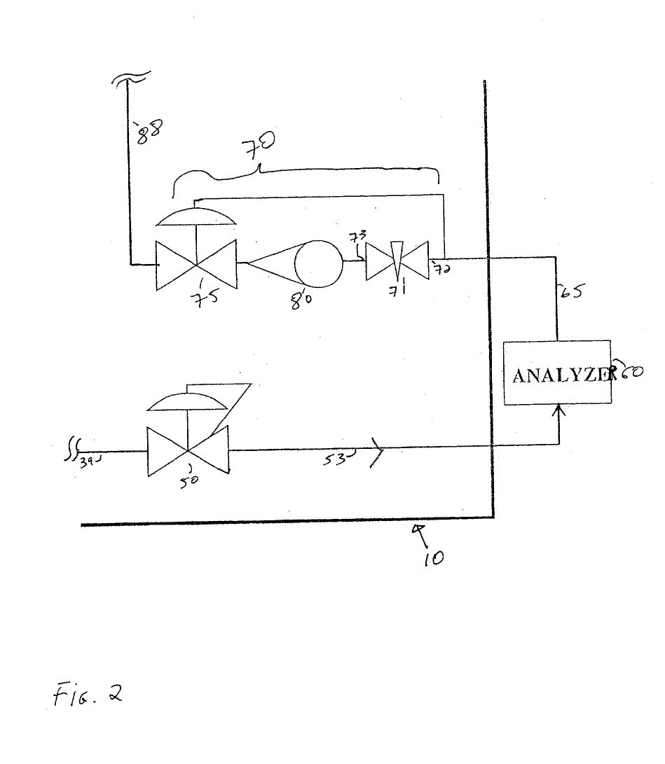

Referring to FIG. 2, pressure-reducing regulator 50 is thus positioned between switching system 30, as shown in FIG. 1, and analyzer measuring cell 60. Gas stream pressures can vary as much as 80 psi leading up to pressure-reducing regulator 50. These varying pressure streams enter regulator 50 and are reduced to a constant predetermined pressure suitable for measurement by analyzer measuring cell 60.

After exiting the pressure-reducing regulator 50, the first sample gas stream is routed to analyzer measuring cell 60. The latter measures the concentration of at least one or more specific constituents or components of the sample gas stream and transmits this information to a control system or a plant computer (not shown). In general, as previously noted, analyzers are considered either continuous or non-continuous, and are used in both industrial and non-industrial applications. A few examples of industrial applications are process control, ambient air monitors and environmental monitors, such as used for measuring automobile exhaust emissions, etc.. The present invention is primarily concerned with continuous analyzers, which require a continuous flow of the gas stream through the measuring cell and produce a continuous analysis of the gas stream. A continuous analyzer can usually measure the concentration of at least one component in the gas stream. Examples of such measured components are Oxygen, Carbon Dioxide, Carbon Monoxide and Nitrogen Oxide in a stack monitor.

An example of a batch or non-continuous analyzer is a gas chromatograph, such as the Advance Maxum™ Gas Chromatograph available from Siemens Applied Automation Inc., located in Bartlesville, OK.. Normally the sample pressure of the gas fluid is equilibrated to atmospheric pressure through switching valves just prior to injecting a gas sample into the gas chromatograph. This sample volume is normally vented to atmosphere because the volume, usually less than 10 cc, is so small. However there are installations that require componentry for a substantially constant pressure since certain gas samples, no matter how small, cannot be vented to atmosphere. In these cases, pressure regulator 50 and flow controller 70 in combination with a non-continuous analyzer 60 can also serve to control the flow rate and pressure of such a sample gas stream.

Upon exiting analyzer cell 60, the sample gas stream enters flow controller 70. Flow controller 70 maintains a substantially constant flow rate through both the pressure-reducing regulator 50 and analyzer cell 60. A constant flow rate is necessary to ensure a substantially stabilized pressure. Downstream pressures can fluctuate due to backpressure resulting from the containment of the gases being disposed of. Harmful gases can no longer be vented to atmosphere after being analyzed, and must be recaptured within the system. Many, even quite expensive, systems have been devised over the years but none have been very successful in controlling the pressure that is sufficiently constant enough for stable analysis.

A typical example of the cause of backpressure follows. A flare stack in a process plant is used for the disposition of unwanted gas streams and for handling plant upsets, as well as emergency situations. The exhaust of the analyzer is often connected to this flare stack system. The pressure of a header feeding the flare stack usually operates at or around 1 psig. When a plant has an upset or emergency situation, this header pressure can exceed 10 psig. Obviously this degree of fluctuation in backpressure would cause major errors in the analyzer. Referring to FIG. 1, an optional pump 92, or aspirator, can be placed downstream of outlet valve 90 to produce a pressure, normally exceeding 15 psig, sufficient to induce flow into a flare header. Whenever the flare system is not used, it is normal to return the analyzed sample to the gas stream that is of less pressure and thus sufficient to produce flow through the pressure/flow control and gas recovery system 10.

Again referring to FIG. 2, varying backpressures will adversely affect the functioning of analyzer cell 60. As mentioned previously, flow controller 70 is comprised of two components, namely needle valve 71 in combination with backpressure sensitive regulator 75. An example of a commercially available flow controller for use in system 10 is the Veriflo SC423XL Low/Flow Controller, manufactured by the Parker Hannifin Corporation of Cleveland, Ohio. The SC423XL controller was specifically designed for air and analyzer sampling systems, such as system 10, which require very low flow rates (less than 10 sccm).

Backpressure sensitive regulator 75 functions as a differential pressure regulator and controls the pressure differential across needle valve 71. The amount of the pressure differential can be easily adjusted by turning an adjustment screw (not shown) on the bottom of the regulator. If the pressure differential across needle valve 71 is constant, the flow will be constant. Needle valve 71 can also be adjusted to deliver various predetermined flow rates, depending on any of the noted gas streams. Pressure-reducing regulator 50 ensures that the sample gas stream has a constant pressure through analyzer measuring cell 60, via a line 65 connecting analyzer measuring cell 60 to a needle valve inlet orifice 72 of needle valve 71. Backpressure sensitive regulator 75 ensures that downstream gas pressures do not adversely affect the pressure at a needle valve outlet orifice 73 and that the pressure at needle valve outlet orifice 73 is held substantially constant. Therefore, due to the utilization of pressure reducing regulator 50 in combination with backpressure regulator 75, the pressure differential across needle valve 71 is held substantially constant.

Pressure reducing regulator 50 and flow controller 70 ensure that the flow rate and pressure remains substantially constant from line 53 through needle valve 71. Analyzer measuring cell 60 is positioned between these noted components and will not be adversely affected by pressure and flow fluctuations outside of this area. Substantially constant pressure and flow rates are necessary for an effective analysis of the gas streams.

Referring again to FIG. 1, upon exit from flow controller 70, the analyzed sample gas stream flows through line 88 and reunites with the by-passed sample gas stream in line 58. This combined sample gas stream then flows through return valve 90 via line 58 and is then routed for proper disposal, as previously described.

As previously noted, it is important to maintain a substantially constant pressure within analyzer measuring cell 60. It is also important that the gas stream flows at a substantially constant rate through analyzer measuring cell 60. Gas stream pressures can vary as much as 80 psi upstream of the analyzer cell 60. With the inclusion of pressure reducing regulator 50, these varying upstream pressures are maintained at a preferred level. These varying downstream pressures, or backpressures, will also negatively affect the function of analyzer measuring cell 60. Flow controller 70, and specifically backpressure sensitive regulator 75, substantially prevents downstream sample return pressure fluctuations from reaching analyzer measuring cell 60. Flow controller 70 also ensures that the gas stream flows at a substantially constant rate through analyzer measuring cell 60. Therefore with the inclusion of pressure reducing regulator 50 and flow controller 70, analyzer cell 60 is substantially isolated from all upstream and downstream pressure and flow rate fluctuations. With a substantially constant pressure and flow rate within analyzer measuring cell 60, the required accuracy of measurements is ensured.

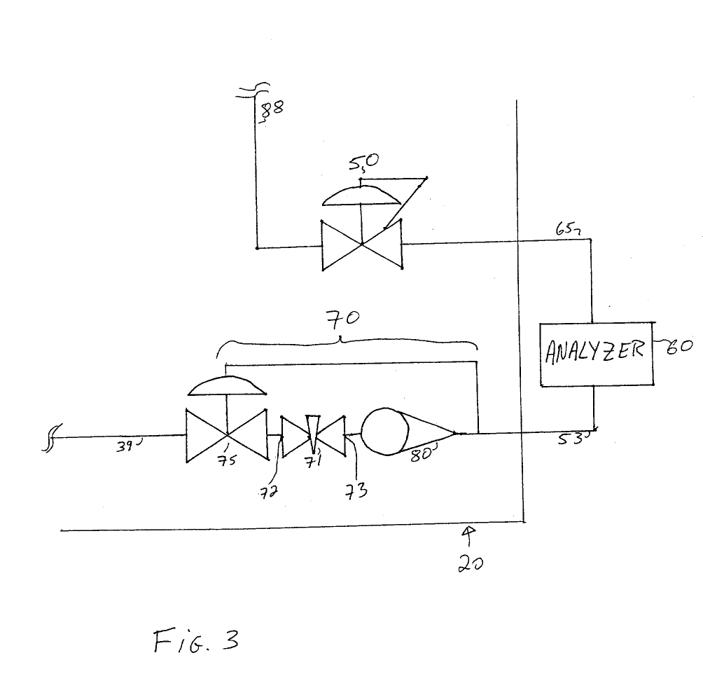

A second embodiment 20 of the present invention is shown in FIG. 3, which is a variation of first embodiment 10, shown in FIGS. 1 and 2. In second embodiment 20, flow controller 70 is positioned upstream of analyzer measuring cell 60 and pressure reducing regulator 50 is positioned downstream of analyzer measuring cell 60. This reversal of the noted locations of flow controller 70 and pressure reducing regulator 50 accomplishes the same goal as in first embodiment 10, which is to provide a substantially constant gas pressure and flow rate with reference to analyzer measuring cell 60. As the single outlet flow stream exits stream switching system 30, as shown in FIG. 1, through line 39, it will enter backpressure regulator 75, which functions as a pressure regulator so that the previously described gas streams enter interconnected needle valve inlet orifice 72 of needle valve 71 at a substantially constant pressure. As previously noted, the pressure differential across needle valve 71 must be held substantially constant. If the pressure differential is substantially constant, the flow rate will be substantially constant. As is the case in the first embodiment 10, flow meter 80 is attached to needle valve 71 and indicates the flow rate of the gas stream exiting needle valve 71.

In the second embodiment 20, the gas stream outlet pressure at needle valve 71 is controlled by pressure regulator 50 which here has been positioned downstream of analyzer measuring cell 60. Pressure regulator 50 is so oriented that downstream gas back-pressures translated through line 88 do not continue through regulator 50, and thus can not affect analyzer measuring cell 60. Thus, pressure reducing regulator 50 provides a substantially constant, predetermined gas pressure at needle valve outlet orifice 73. With a substantially constant flow rate, and pressure, through needle valve 71, the stream pressure and flow rate in line 53 will also be substantially constant. Thus, analyzer measuring cell 60 will not be subjected to any substantial pressure or flow rate fluctuations and will be able to function at the prescribed operating conditions, i.e. at substantially constant pressure and flow rates, at all times.

What is claimed is:

1. A method of maintaining both a constant pressure rate and a constant flow rate for a sample gas stream flowing through a gas analyzer measuring cell, said gas analyzer measuring cell being operatively interposed in a closed system between a flowing pressurized gas stream and a gas recovery system, said method comprising the steps of:
a. continuously extracting a sample gas stream from said gas stream;
b. directing at least a portion of said sample gas stream into and through said analyzer measuring cell;
c. controlling the pressure of said sample gas stream for operating said gas analyzer measuring cell at a substantially constant value;
d. controlling the flow rate of said sample gas stream so that said flow rate remains at a substantially constant value while said sample gas stream passes through said gas analyzer measuring cell;
e. analyzing said sample gas stream for at least one constituent thereof, while under said substantially constant pressure and flow rate values, as said sample gas stream passes through said gas analyzer measuring cell; and
f. directing said analyzed sample gas stream to said gas recovery system.

2. The method according to claim 1, further including the following successive steps before step a. of claim 1:
a. extracting a zero gas stream from a zero gas reservoir and directing said zero gas stream into and through said gas analyzer measuring cell until the operation of said cell has been stabilized;
b. controlling the pressure of said zero gas stream for operating said gas analyzer measuring cell at a substantially constant value;
c. controlling the flow rate of said zero gas stream so that said flow rate remains at a substantially constant value while said zero gas stream passes through said gas analyzer measuring cell;
d. extracting a calibration gas stream from a calibration gas reservoir and directing said calibration gas stream into and through said gas analyzer measuring cell until said cell has been calibrated;
e. controlling the pressure of said calibration gas stream for operating said gas analyzer measuring cell at a substantially constant value; and
f. controlling the flow rate of said calibration gas stream so that said flow rate remains at a substantially constant value while said calibration gas stream passes through said gas analyzer measuring cell.

3. The method according to claim 2, further including the additional steps of successively segregating said zero gas stream, said calibration gas stream and said sample gas stream from each other while flowing to said gas analyzer measuring cell.

4. The method according to claim 1, including the additional steps of:
a. directing said sample gas stream from said gas stream to a filter assembly;
b. separating said sample gas stream into a first sample gas stream, and filtering same, and a second sample gas stream and by-passing said filtering step;
c. directing said filtered first sample gas stream into and through said gas analyzer measuring cell; and
d. directing said by-passed second sample gas stream into said gas recovery system.

5. The method according to claim 2, including the additional steps of:
a. directing said sample gas stream from said gas stream to a filter assembly;
b. separating said sample gas stream into a first sample gas stream, and filtering same, and a second sample gas stream and by-passing said filtering step;
c. directing said filtered first sample gas stream into and through said gas analyzer measuring cell; and
d. directing said by-passed second sample gas stream into said gas recovery system.

6. The method according to claim 4, wherein in said separating step, the volume of said first sample gas stream is less than 25% of the volume of said sample gas stream.

7. The method according to claim 4, including the additional step of varying the volume of said by-passed second sample gas stream prior to the entering of said by-passed second sample gas stream into said gas recovery system.

8. The method according to claim 7, including the additional step of varying the volume of said first sample gas stream by varying the volume of said second sample gas stream.

9. In an apparatus for successively removing a portion of a flowing effluent gas stream as a sample gas stream, analyzing said sample gas stream for at least one constituent thereof and thereafter returning said analyzed sample gas stream to a gas recovery system, a closed gas analyzing system comprising:
a. a plurality of differing pressurized gas sources, including a zeroing gas stream, calibration gas stream and said sample gas stream;
b. a filter assembly operatively interconnected with said sample gas stream, said filter assembly including a stream separator for separating said sample gas stream into a first sample gas stream for filtering same and a second sample gas stream for by-passing said filtering, said filter assembly further including filtering means for said filtering said first sample gas stream, said by-passed second sample gas stream being operatively interconnected with said gas recovery system;
c. a stream switching manifold device operatively interconnected with said sources of said zeroing gas stream, said calibration gas stream and said first sample gas stream, said stream switching manifold device having but one outlet, said stream switching manifold device further including a stream selector for selectively segregating said zeroing gas stream, said calibration gas stream and said first sample gas stream;
d. a gas analyzer measuring cell successively operatively interconnected with the outlet of said stream switching manifold device and said by-passed second sample gas stream;
e. a pressure regulator, operatively interposed between the outlet of said stream switching manifold device and said gas analyzer measuring cell, for successively controlling the pressures of said zeroing gas stream, said calibration gas stream and said first sample gas stream such that said controlled pressures are substantially constant upon the exits of said gas streams from said pressure regulator; and
f. a flow controller, operatively interposed between said gas analyzer measuring cell and said gas recovery system for both
successively regulating the flow rates of said zeroing gas stream, said calibration gas stream and said filtered first sample gas stream, flowing through said gas analyzer measuring cell, to a substantially constant value; and
substantially preventing backpressure variations from entering said gas analyzer measuring cell from said gas recovery system.

10. The system of claim 9 wherein said gas analyzer measuring cell utilizes a continuously operating analyzer.

11. The system of claim 9 wherein said gas analyzer measuring cell utilizes a non-continuously operating analyzer.

12. The system of claim 9 wherein the volume of said second sample gas stream is greater than 75% of the volume of said sample gas stream.

13. The system of claim 9 wherein said flow controller is comprised of a needle valve operatively interconnected with a backpressure regulator.

14. The system of claim 13 wherein said needle valve is adjustable for varying the volume said zeroing gas stream, said calibration gas stream, and said filtered first sample gas stream.

15. The system of claim 9 including a further adjustable needle valve, operatively interposed in said second sample gas stream, for varying the volume of said by-passed second sample gas stream prior to entering said gas recovery system.

16. In an apparatus for successively removing a portion of a flowing gas stream as a sample gas stream, analyzing said sample gas stream for at least one constituent thereof and thereafter returning said analyzed sample gas stream to a gas disposal system, a pressure/flow control and gas recovery system comprising:
a. plurality of differing pressurized gas sources, including a zeroing gas stream, a calibration gas stream and said sample gas stream;
b. an individual control valve operatively interconnected for each of said sources of said zeroing gas stream, said calibration gas stream and said sample gas stream controlling the flow of said zeroing gas stream, said calibration gas stream and said gas sample stream;
c. a gas analyzer measuring cell successively operatively interconnected with said individual control valves and said gas disposal system;
d. a pressure regulator, operatively interconnected with said gas analyzer measuring cell, for successively controlling the pressures of said zeroing gas stream, said calibration gas stream and said sample gas stream such that said controlled pressures are substantially constant upon the exits of said gas streams from said pressure regulator; and
e. a flow controller, operatively interconnected with said gas analyzer measuring cell for both
successively regulating the flow rates of said zeroing gas stream, said calibration gas stream and said sample gas stream, flowing through said gas analyzer measuring cell, to a substantially constant value; and substantially preventing backpressure variations from entering said gas analyzer measuring cell from said gas disposal system.

17. The system of claim 16 further including a filter assembly operatively interconnected with said sample gas stream, said filter assembly including a stream separator for separating said sample gas stream into a first sample gas stream for filtering same and a second sample gas stream for by-passing said filtering, said filter assembly further including filtering means for said filtering said first sample gas stream, said by-passed second sample gas stream being operatively interconnected with said gas disposal system.

18. The system of claim 16 further including, in lieu of said individual control valves, a stream switching manifold device operatively interconnected with said sources of said zeroing gas stream, said calibration gas stream and said sample gas stream, said stream switching manifold device having but one outlet, said stream switching manifold device further including a stream selector for selectively segregating said zeroing gas stream, said calibration gas stream and said gas sample stream.

19. The system of claim 16 wherein said gas analyzer measuring cell utilizes a continuously operating analyzer.

20. The system of claim 16 wherein said gas analyzer measuring cell utilizes a non-continuously operating analyzer.

21. The system of claim 17 wherein the volume of said second sample gas stream is greater than 75% of the volume of said sample gas stream.

22. The system of claim 17 further including an adjustable needle valve, operatively interposed in said second sample gas stream, for varying the volume of said second sample gas stream prior to entering said gas disposal system.

23. The system of claim 16 wherein said flow controller is comprised of an additional needle valve operatively interconnected with a backpressure regulator.

24. The system of claim 23 wherein said additional needle valve is adjustable for varying the volume of said zeroing gas stream, said calibration gas stream, and said first sample gas stream.

25. The system of claim 16 wherein said pressure regulator is operatively interposed between the outlet of said individual control valves and said gas analyzer measuring cell.

26. The system of claim 18 wherein said pressure regulator is operatively interposed between the outlet of said stream switching manifold device and said gas analyzer measuring cell.

27. The system of claim 16 wherein said pressure regulator is operatively interposed between said gas analyzer measuring cell and said gas disposal system.

28. The system of claim 16 wherein said flow controller is operatively interposed between said gas analyzer measuring cell and said gas disposal system.

29. The system of claim 16 wherein said flow controller is operatively interposed between said outlet of said individual control valves and said gas analyzer measuring cell.

30. The system of claim 18 wherein said flow controller is operatively interposed between said outlet of said stream switching manifold device and said gas analyzer measuring cell.

31. The system of claim 16 further including a pump or aspirator operatively interposed in said pressure/flow control and vent recovery system upstream of said gas disposal system.

32. A method of maintaining both a constant pressure rate and a constant flow rate for a sample gas stream flowing through a gas analyzer measuring cell, said gas analyzer measuring cell being operatively interposed in a system between a flowing pressurized gas stream and a gas recovery system, said method comprising the steps of:
a. directing said sample gas stream, from said gas stream, into and through said analyzer measuring cell;
b. controlling the value of the pressure of said sample gas stream for operating said gas analyzer measuring cell at a substantially constant value;
c. controlling the value of the flow rate of said sample gas stream so that said flow rate remains at a substantially constant value while said sample gas stream passes through said gas analyzer measuring cell;
d. analyzing said sample gas stream for at least one constituent thereof, while under said substantially constant pressure and flow rate values, as said sample gas stream passes through said gas analyzer measuring cell; and
e. directing said analyzed sample gas stream to said gas recovery system.

33. The method according to claim 32, further including the following steps before step a. of claim 32:
a. directing a zero gas stream, from a zero gas source, into and through said gas analyzer measuring cell for stabilizing the operation of said cell;
b. directing a calibration gas stream, from a calibration gas source, into and through said gas analyzer measuring cell for calibrating said cell;
c. controlling the value of the pressures of said zero gas stream and said calibration gas stream for operating said gas analyzer measuring cell at a substantially constant value; and
d. controlling the value of the flow rates of said zero gas stream and said calibration gas stream so that said flow rate remains at a substantially constant value while said zero gas stream and said calibration gas stream pass through said gas analyzer measuring cell.

34. The method according to claim 33, further including the steps of segregating said zero gas stream, said calibration gas stream and said sample gas stream from each other before flowing to said gas analyzer measuring cell.

35. The method according to claim 32, comprising the additional steps of:
a. directing said sample gas stream from said gas stream to a filter assembly;
b. separating said sample gas stream into a first sample gas stream, and filtering same, and a second sample gas stream and by-passing said filtering step;
c. directing said filtered first sample gas stream into and through said gas analyzer measuring cell; and
d. directing said by-passed second sample gas stream into said gas recovery system.

36. The method according to claim 35, wherein in said separating step, the volume of said second sample gas stream is greater than 75% of the volume of said sample gas stream.

37. The method according to claim 35, including the additional step of varying the volume of said by-passed second sample gas stream prior to the entering of said by-passed second sample gas stream into said gas recovery system.
